(19) United States
(12) Patent Application Publication
(10) Pub. No.: US 2025/0313603 A1
(43) Pub. Date: Oct. 9, 2025

(54) Primary NK CAR Constructs And Methods

(71) Applicant: ImmunityBio, Inc., San Diego, CA (US)

(72) Inventors: Fereshteh Parviz, San Diego, CA (US); Rohit Duggal, San Diego, CA (US)

(73) Assignee: ImmunityBio, Inc., San Diego, CA (US)

(21) Appl. No.: 19/246,845

(22) Filed: Jun. 24, 2025

Related U.S. Application Data
(62) Division of application No. 17/311,226, filed on Jun. 4, 2021.

(51) Int. Cl. C07K 14/55; A61K 40/15; A61K 40/31; A61K 40/42; A61K 45/06; C07K 14/705; C07K 14/725; C07K 16/28; C12N 5/0783; C12N 15/85

(52) U.S. Cl. CPC C07K 14/55; A61K 40/15; A61K 40/31; A61K 40/421; A61K 40/4234; A61K 45/06; C07K 14/7051; C07K 14/70521; C07K 16/2827; C12N 5/0638; C12N 5/0646; C12N 15/85; C07K 2317/622; C07K 2317/732; C07K 2319/03; C07K 2319/33; C12N 2510/00; C12N 2830/001; C12N 2830/50; C12N 2840/203

(57) ABSTRACT

Disclosed herein are recombinant nucleic acids, comprising a 5′ untranslated (5′-UTR) sequence portion, a signal peptide sequence portion, a single chain antibody fragment sequence portion, a hinge region sequence portion, a transmembrane domain sequence portion, and one or more intracellular domain sequence portions. Also disclosed herein are modified natural killer (NK) cells comprising the recombinant nucleic acid described above. Further disclosed herein are methods of treating a tumor in a subject by administering the modified NK cells.

This application is a Divisional of U.S. patent application Ser. No. 17/311,226, filed Jun. 4, 2021, which is a national phase application of PCT Application No. PCT/US2019/063454, filed on Nov. 26, 2019. Each of these applications are incorporated by reference herein in its entirety.

### SEQUENCE LISTING

The content of the XML file of the sequence listing named 104077.00016US2, which is 38,006 bytes in size was created on Jun. 4, 2025 and electronically submitted via EFS-Web along with the present application is incorporated by reference in its entirety.

### FIELD OF THE INVENTION

The field of the invention is recombinant nucleic acids and cells containing the same, particularly as they relate to the treatment of cancer.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

Cancer immunotherapies based on natural killer (NK) cells have had remarkable progress in recent years. NK cells are cytotoxic lymphocytes that constitute a significant component of the innate immune system. In most cases, NK cells represent about 4-10% of circulating lymphocytes, and bind and kill targeted cells, including virus-infected cells and many malignant cells. NK cell killing is non-specific with regard to particular antigens and can occur without prior immune sensitization. Killing of targeted cells is typically mediated by cytolytic proteins, including perforin, granzyme, and granulysin.

NK cells have been used as therapeutic entities. To that end, NK cells are isolated from the peripheral blood lymphocyte fraction of whole blood, expanded in cell culture to obtain sufficient numbers of cells, and then re-infused into a subject. NK cells have shown in at least some cases moderate effectiveness in both ex vivo therapy and in vivo treatment. However, cancers employ various tactics to delay, alter, or even stop anti-tumor immunity, leading to failures in the control of tumor growth.

The anti-tumor response of NK cells also faces a lot of limitations. First, the poor ability of NK cells to reach tumor tissues limits their application as therapies for solid tumors. This is a common problem of cellular immunotherapy strategies. Second, changes in NK cell-activating receptors and their ligands in tumors, may lead to a decreased therapeutic response and tumor progression. For example, high levels of NKG2D (Natural-killer Group 2, Member D) ligands are detected in the early stages of colorectal cancer, but their expression decreases as the disease progresses. Third, the tumor microenvironment (TME) remains a major barrier to the effectiveness of adoptively transferred NK cells. For example, tumor-infiltrating immune cells such as dendritic cells (DCs), suppressive or tolerogenic macrophages and regulatory T (Treg) cells as well as cancer-associated fibroblasts, which are embedded in the extracellular matrix, may meddle in NK cell activation either through secretion of immunosuppressive cytokines or by interfering with receptor expression.

Thus there remains a need in the art for technologies and methods for overcome the above problems and being able modify NK cells for specific targeting of cancer cells.

### SUMMARY OF THE INVENTION

The inventive subject matter is directed to recombinant nucleic acids, comprising a T7 promoter sequence portion, a 5′ untranslated (5′-UTR) sequence portion, a signal peptide sequence portion, a single chain antibody fragment sequence portion, a hinge region sequence portion, a transmembrane domain sequence portion, and one or more intracellular domain sequence portions. The recombinant nucleic acid may further comprises a sequence encoding CD64. Furthermore, the 5′-UTR sequence portion may further comprise a kozak sequence.

Preferably, the single chain antibody fragment sequence portion comprises a sequence encoding for a single chain variable fragment that is adapted to bind PDL1 antigen or other tumor antigens. In some embodiment, the recombinant nucleic acid may further comprise a sequence portion encoding CD16a and/or ER-IL2.

The hinge sequence portion provides range of motion for the single chain antibody fragment sequence portion, while the transmembrane domain sequence portion enables insertion of the recombinant nucleic acid to a membrane.

The intracellular domain sequence portion of the recombinant nucleic acid as disclosed herein is contemplated to comprise co-stimulatory or signaling sequence portions. In one embodiment, the intracellular domain sequence portion comprises CD28 and/or CD3ζ. In another embodiment, the intracellular domain sequence portion comprises CD28 and/or FcεRIγ. The recombinant nucleic acid of any one of the preceding claims, Furthermore, the intracellular domain sequence portion may provide enhanced cytotoxic activity against tumor cells.

Preferably, the recombinant nucleic acid of this disclosure comprises a 3′-untranslated region (3′-UTR) and a poly-A sequence portion. The 3′-UTR sequence portion provides RNA stability and initiation of translation. The poly-A sequence portion preferably comprises at least 150 adenine nucleotides. The poly-A sequence portion provides RNA stability and initiation of translation.

The recombinant nucleic acid vector of this disclosure is preferably optimized to target a tumor antigen. In some embodiments, the recombinant nucleic acid has at least 90%, 91%, 92%, 93%, 94%, 95%, 96%, 97%, 98% or 99% identity to SEQ ID NO: 1. In other embodiments, the recombinant nucleic acid has at least 90%, 91%, 92%, 93%, 94%, 95%, 96%, 97%, 98% or 99% identity to SEQ ID NO: 2. In still other embodiments, the recombinant nucleic acid, has at least 90%, 91%, 92%, 93%, 94%, 95%, 96%, 97%, 98% or 99% identity to SEQ ID NO: 3. In still further embodiments, the recombinant nucleic acid, has at least 90%, 91%, 92%, 93%, 94%, 95%, 96%, 97%, 98% or 99% identity to SEQ ID NO: 4.

In another aspect of the inventive subject matter, the inventors have disclosed a modified NK cell comprising one or more nucleic acids encoding: a 5′ untranslated (5′-UTR) sequence portion, a signal peptide sequence portion, a single chain antibody fragment sequence portion, a hinge region sequence portion, a transmembrane domain sequence portion, and one or more intracellular domain sequence portions; wherein the nucleic acid sequences are operably linked to each other as a single polynucleotide. This modified NK cell is contemplated to specifically target tumor cells.

In another aspect, the inventors have disclosed a method of generating modified NK cells or CAR-NK cells, comprising: transfecting a primary NK cell with a recombinant nucleic acid as disclosed above. Furthermore, a composition is also disclosed comprising the modified NK cell and a pharmaceutically acceptable excipient. Furthermore, the modified NK cell may be provided in a kit; for example the kit may comprise the NKcell as disclosed herein and instructions for use.

In yet another aspect, disclosed is a method of treating a cancer or a tumor in a subject, the method comprising administering to the subject a therapeutically effective amount of the modified NK cells or the composition comprising the modified NK cells, wherein administration treats the cancer or reduces the size of the tumor in the subject. A method of reducing cancer metastasis in a patient is also contemplated, wherein a subject having cancer metastasis is administered with a therapeutically effective amount of the modified NK cells or a composition comprising modified NK cells. Preferably, from 1×10³ to 1×10¹⁰, per m² of the NK cells are administered to the subject. The administration may be done parenterally, intravenously, peritumorally, or by infusion. The method may also comprise further administration to the subject an additional therapeutic agent.

In another embodiment, the inventors have disclosed a method of treating cancer in a patient in need thereof, comprising administering to the patient a therapeutically effective amount of any one of the genetically modified NK cells as disclosed herein, thereby treating the cancer. The method may further comprise a step of administering at least one additional therapeutic entity selected from the group consisting of a viral cancer vaccine, a bacterial cancer vaccine, a yeast cancer vaccine, N-803, an antibody, a stem cell transplant, and a tumor targeted cytokine. The cancer is selected from leukemia, acute lymphocytic leukemia, acute myelocytic leukemia, chronic leukemias, chronic myelocytic (granulocytic) leukemia, chronic lymphocytic leukemia, polycythemia vera, lymphomas, Hodgkin's disease, non-Hodgkin's disease, multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 depicts one embodiment of generation of a chimeric antigen targeted at PDL1 protein: pNBS-XL53.

FIG. 2 shows one embodiment of the time course of XL53 PDL1 CAR expression post electroporation.

FIG. 3 illustrates one embodiment of the cytotoxic activity on fLuc expressing U251 and MS1 target cells.

FIG. 4 shows one embodiment of a vector map of the XL53 construct.

FIG. 5 shows one embodiment of generation of a chimeric antigen targeted at PDL1 protein, pNBS-XL53-150A.

FIG. 6 illustrates one embodiment of a time course of XL53-150A PDL1 CAR expression post electroporation.

FIG. 7 illustrates one embodiment of cytotoxic activity of NK cells transfected with XL53-150A.

FIG. 8 shows one embodiment of a vector map of the XL53-150A construct.

FIG. 9 shows one embodiment of generation of a chimeric antigen targeted at PDL1 protein, NKW29-150A.

FIG. 10 depicts one embodiment of time course of NKW29-150A PDL1 CAR expression in NK cells.

FIG. 11 illustrates one embodiment of a vector map of the NKW29-150A construct.

FIG. 12 illustrates one embodiment of generation of a chimeric antigen targeted at PDL1 protein, tricistronic XL35.

FIG. 13 illustrates one embodiment of XL53-tricistronic PDL1 CAR expression 24 hours post electroporation.

FIG. 14 depicts one embodiment of cytotoxic activity of CAR-infected cells on MS1 fLuc target cells.

FIG. 15 illustrates one embodiment of vector map of the XL53-tricistronic construct.

### DETAILED DESCRIPTION

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply. Unless defined otherwise, all technical and scientific terms used herein have the same meaning as commonly understood by one of ordinary skill in the art to which this invention belongs.

The inventors have disclosed herein various engineered NK cells as the basis to improve immunotherapies to cancer and tumors. Viewed from a different perspective, the inventors have disclosed nucleic acid constructs that target tumor antigens. Preferably, in some embodiments, the tumor antigen is PDL1. In one embodiment, these nucleic acid constructs may be chimeric antigen receptor constructs for transfecting primary NK cells to generate CAR-NK cells.

In one aspect of the inventive concept, the disclosure herein involves generation of a chimeric antigen RNA molecule (CAR) against PDL1 and potentially other tumor antigen targets. The RNA generated from these type of DNA constructs is contemplated to be delivered to natural killer cells for specific targeting of tumor cells.

In one embodiment, disclosed herein is a recombinant nucleic acid, comprising: a T7 promoter sequence portion, a 5′ untranslated (5′-UTR) sequence portion, a signal peptide sequence portion, a single chain antibody fragment sequence portion, a hinge region sequence portion, a transmembrane domain sequence portion, and one or more intracellular domain sequence portions. In one embodiment, the recombinant nucleic acid comprises or consists of or consists essentially of an amino acid sequence having at least about 80%, 85%, 90%, 95%, 96%, 97%, 98%, or 99% sequence homology to the nucleotide sequence of SEQ ID NOs: 1-4.

The recombinant nucleic acid construct of SEQ ID NO: 1, also referred to herein as XL53, is targeted against a PD1 ligand called PDL1. This molecule is designed for in vitro synthesis of an RNA molecule that would be delivered to natural killer cells for the purpose of immunotherapy in cancer patients. In vitro transcription can be initiated at the T7 promoter using the bacteriophage T7 RNA polymerase. The T7 promoter is flanked by a 42-bp untranslated sequence (5′ UTR: 5′ untranslated) that also contains a Kozak sequence upstream of the CAR gene. The secondary structure of the 5′ UTR along with the Kozak sequence aid with the initiation of translation. A short signal peptide (15-amino acids) from the CD64 protein, marks the N-terminus of the CAR protein. The signal peptide is recognized by a signal recognition peptide (SRP) in the cytosol that delivers the nascent polypeptide chain from the cytosol to the endoplasmic reticulum. The PDL1 binding site is a heterodimer of variable light and heavy chain domains. The two domains are connected to each other via a 20-aa (amino acids) linker. This hinge and transmembrane domains of the molecule are derived from the CD28 protein. The hinge region provides range of motion and flexibility for the binding domain while the transmembrane region allows correct membrane insertion. The cytoplasmic domains of CD28 and CD35 are co-stimulatory domains engaged in intracellular signaling pathways that enhance cytotoxic activities of the transfected cells. At the 3′ untranslated end of the construct, a 94-bp sequence from 3′ UTR of Mus musculus hemoglobin alpha gene, further stabilizes the construct. This 3′UTR is followed by 22-bp polyA stretch. The combination of the 3′ UTR and poly A confer stability to the RNA molecule. The main features of the construct of SEQ ID NO: 1 are that (a) it has a high binding affinity for the PDL1 protein; (b) it uses a combination of intracellular domains of CD28 and CD3ζ for enhanced cytotoxic activity against target cells; and (c) it is RNA based, so there is no concern regarding integration of the construct into the host genome.

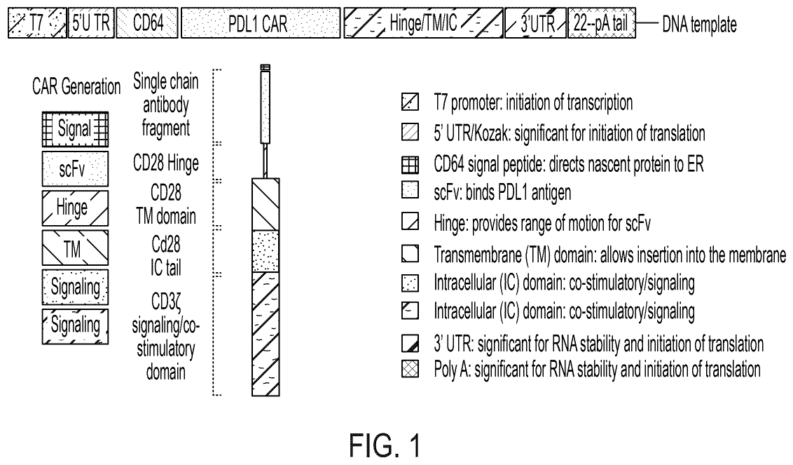

FIG. 1 illustrates generation of a chimeric antigen targeted at PDL1 protein. The transcription of RNA is initiated by the T7 promoter. The 5′-UTR/Kozak region of nucleotides provides significant for initiation of translation. The nucleic acid sequence encoding he CD64 signal peptide is present in the 3′ end of the UTR/Kozak region, and it directs nascent protein to ER. This is followed by scFv region, which binds PDL1 antigen. A hinge region is present next to the scFv region, and that provides a range of motion to the scFv. The hinge region is followed by a transmembrane domain that allows insertion of the nucleotide construct into the membrane. This is followed by one or more intracellular domains comprising co-stimulatory and/or signaling elements. Finally, the 3′-UTR and Poly-A regions are present to provide stability to the RNA as well as initiation of translation.

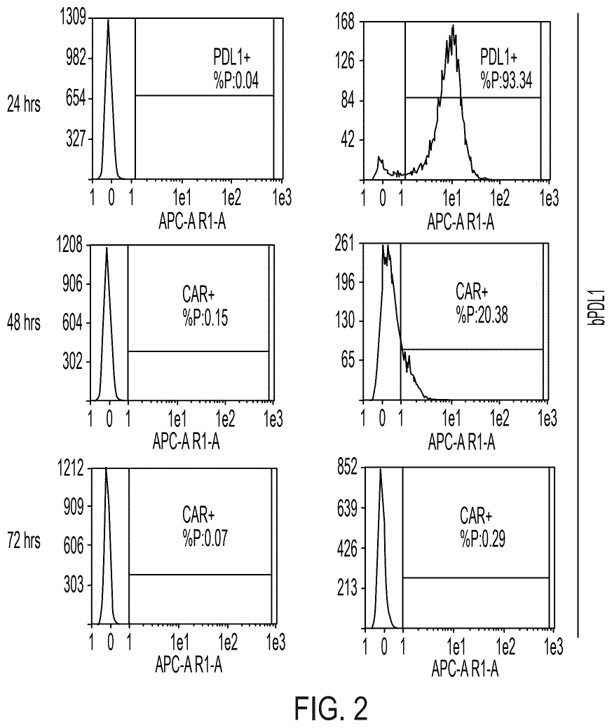

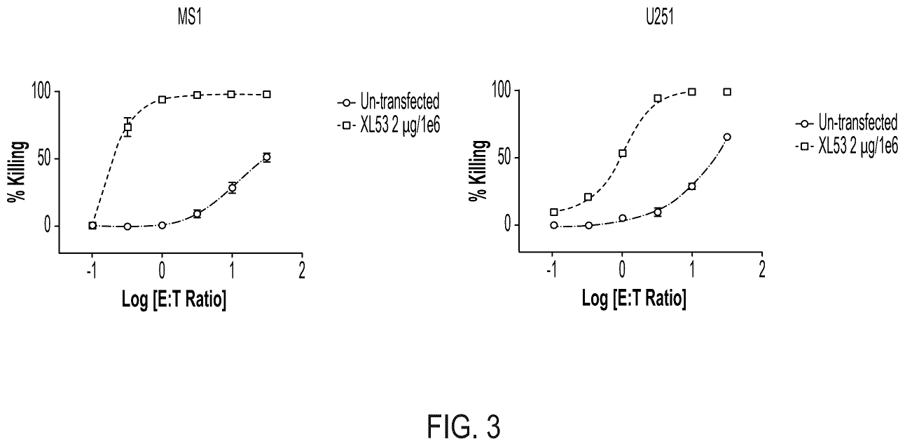

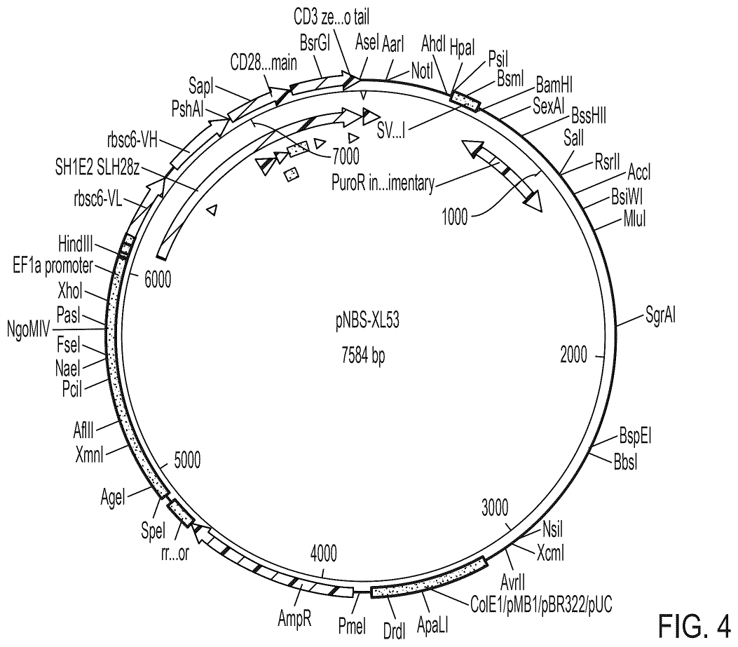

FIG. 2 illustrates time course of XL53 PDL1 CAR expression post electroporation, while the cytotoxic activity on fLuc expressing U251 and MS1 target cells are shown in FIG. 3. The cytotoxic assay, as illustrated in FIG. 3, was set up to 2 hours post transfection and after overnight incubation. The vector map of the XL53 construct is shown in FIG. 4. Finally, Table 1 below shows the different sequence portions of the XL53 construct.

TABLE 1

| | pNBS-XL-53(Rbsc6- |
|---|---|
| | CD28CD3z-anti-PDL1) |
| Size | 7584 bp |
| 3′UTR Mus Musculus | 1-94 |
| hemoglobin alpha | |
| polyA | 95-116 |
| SV40 polyA signal | 433-567 |
| Puromycin resistance gene | 572-1171 |
| (reverse) | |
| OriP | 3187-3753 |
| Ampicillin resistance gene | 3842-4702 |
| rrnG terminator | 4700-4835 |
| EF1a promoter | 4879-6060 |
| T7 promoter | 6068-6086 |
| 5′UTR | 6087-6192 |
| Kozak sequence | 6123-6130 |
| CD64 signal peptide | 6132-6176 |
| Rbsc6-VL (variable light | 6177-6500 |
| chain) | |
| Linker | 6501-6560 |
| Rbsc6-VH (variable heavy | 6561-6914 |
| chain) | |
| CD28 hinge region | 6915-7031 |
| CD28 transmembrane domain | 7032-7112 |
| CD28 cytoplasmic domain | 7113-7235 |
| CD3ζ cytoplasmic domain | 7236-7577 |

In another embodiment, the inventors have disclosed the molecule XL53-150A, which comprises the recombinant nucleic acid construct of SEQ ID NO: 2. This molecule is similar to the XL53 molecule except for the following modifications: A 150-polyA stretch is added to the 3′ untranslated end of the construct to further stabilize the RNA molecule. Also an internal SapI restriction site is removed from the construct, while the same site is added at the end of the poly A tail. After linearization of the DNA template with SapI, only A nucleotides remain. This further enhances translation of the RNA molecule. The main features of this construct are that it has a longer poly A tail, and it has a more prolonged half-life compared to XL53.

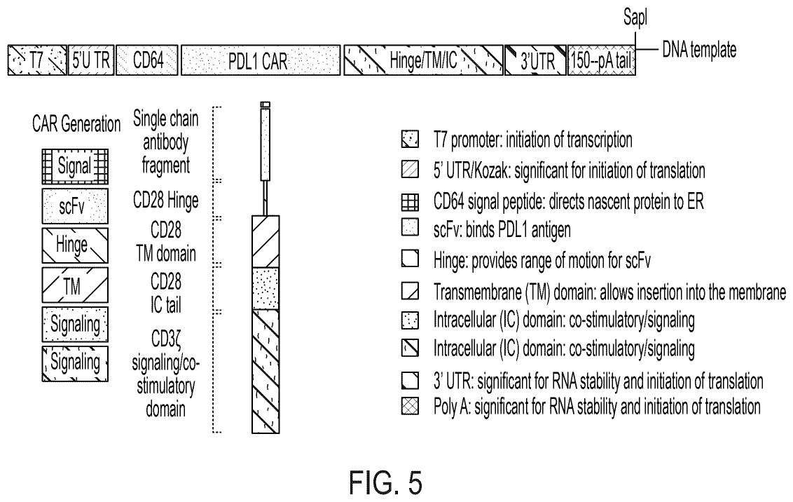

FIG. 5 illustrates generation of a chimeric antigen targeted at PDL1 protein, pNBS-XL53-150A. Similar to the discussion in FIG. 1, the transcription of RNA is initiated by the T7 promoter. The 5′-UTR/Kozak region of nucleotides provides significant for initiation of translation. The nucleic acid sequence encoding the CD64 signal peptide is present in the 3′ end of the UTR/Kozak region, and it directs nascent protein to ER. This is followed by scFv region, which binds PDL1 antigen. A hinge region is present next to the scFv region, and that provides a range of motion to the scFv. The hinge region is followed by a transmembrane domain that allows insertion of the nucleotide construct into the membrane. This is followed by one or more intracellular domains comprising co-stimulatory and/or signaling elements. Finally, the 3′-UTR and Poly-A regions are present to provide stability to the RNA as well as initiation of translation. The longer poly-A region in this construct provides for an RNA construct with more stability and longer half-life time.

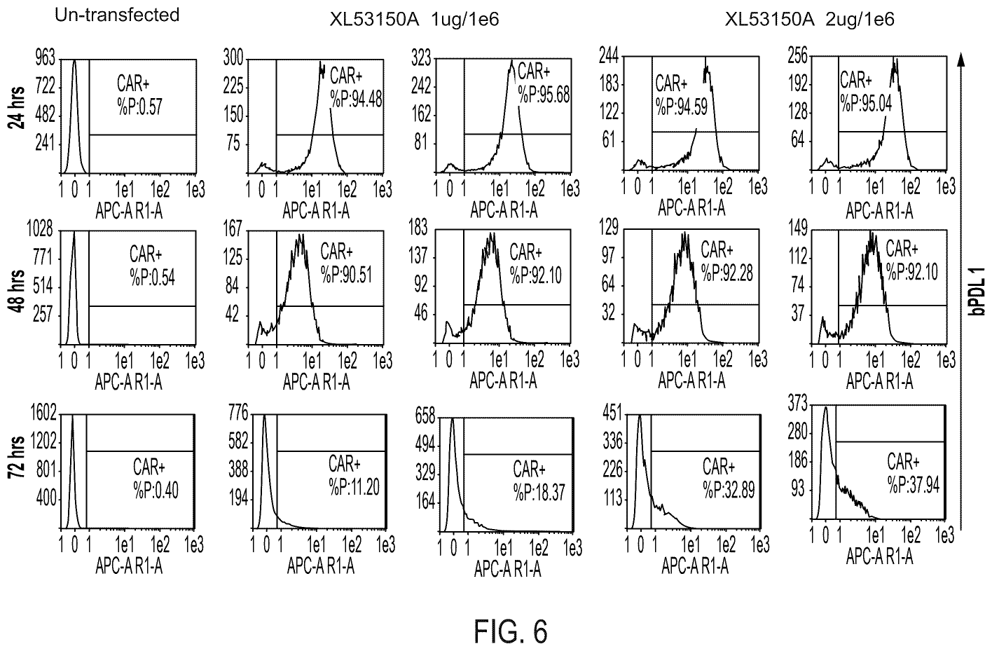

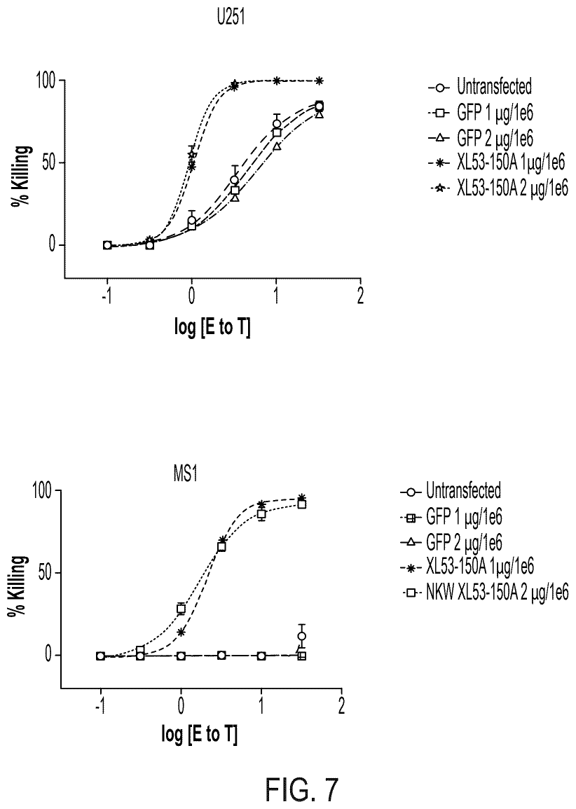

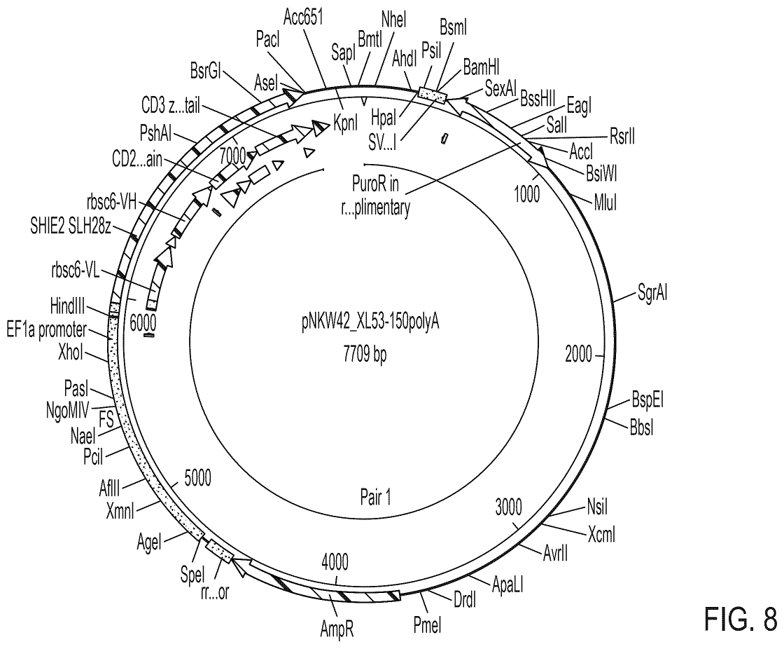

FIG. 6 illustrates time course of XL53-150A PDL1 CAR expression post electroporation, while the cytotoxic activity of NK cells transfected with XL53-150A are shown in FIG. 7. The vector map of the XL53-150A construct is shown in FIG. 8. Finally, Table 2 below shows the different sequence portions of the XL53-150A construct.

TABLE 2

| | pNBS-XL-53(Rbsc6- |
|---|---|
| Construct name | CD28CD3z-anti-PDL1) |
| Size | 7584 bp |
| 3′UTR Mus Musculus | 1-94 |
| hemoglobin alpha | |
| polyA | 95-116 |
| SV40 polyA signal | 433-567 |
| Puromycin resistance gene | 572-1171 |
| (reverse) | |
| OriP | 3187-3753 |
| Ampicillin resistance gene | 3842-4702 |
| rrnG terminator | 4700-4835 |
| EF1a promoter | 4879-6060 |
| T7 promoter | 6068-6086 |
| 5′UTR | 6087-6192 |
| Kozak sequence | 6123-6130 |
| CD64 signal peptide | 6132-6176 |
| Rbsc6-VL (variable light | 6177-6500 |
| chain) | |
| Linker | 6501-6560 |
| Rbsc6-VH (variable heavy | 6561-6914 |
| chain) | |
| CD28 hinge region | 6915-7031 |
| CD28 transmembrane domain | 7032-7112 |
| CD28 cytoplasmic domain | 7113-7235 |
| CD3zeta cytoplasmic domain | 7236-7577 |

In another embodiment, the inventors have disclosed the molecule NKW29, which comprises the recombinant nucleic acid construct of SEQ ID NO: 3. This molecule is very similar to the XL53-150A construct except for the following modification: The CD3ζ intracellular domain of the XL53-150A is replaced with the intracellular domain of FcεRIγ. The main features of this construct are (i) It uses a combination of intracellular domains of CD28 and FcεRIγ for enhanced cytotoxic activity against target cells; and (ii) it is relatively stable due to the long poly A tail.

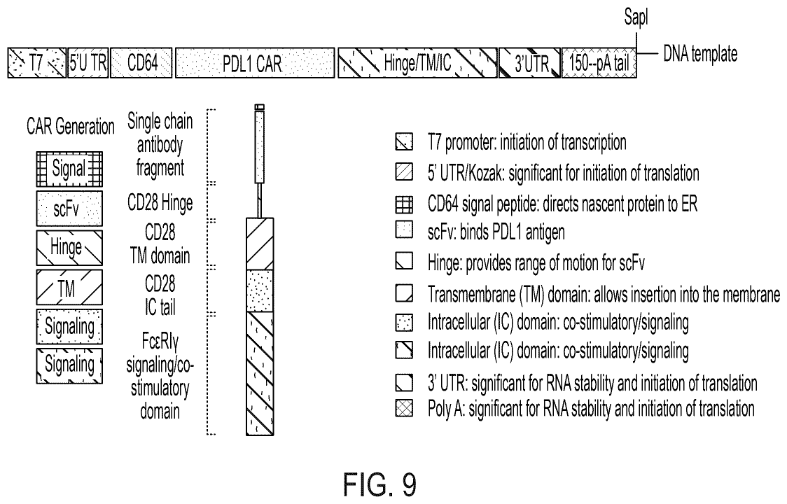

FIG. 9 illustrates generation of a chimeric antigen targeted at PDL1 protein, NKW29-150A. Similar to the discussion in FIGS. 1 and 5, the transcription of RNA is initiated by the T7 promoter. The 5′-UTR/Kozak region of nucleotides provides significant for initiation of translation. The nucleic acid sequence encoding the CD64 signal peptide is present in the 3′ end of the UTR/Kozak region, and it directs nascent protein to ER. This is followed by scFv region, which binds PDL1 antigen. A hinge region is present next to the scFv region, and that provides a range of motion to the scFv. The hinge region is followed by a transmembrane domain that allows insertion of the nucleotide construct into the membrane. This is followed by one or more intracellular domains comprising co-stimulatory and/or signaling elements. The intracellular domain illustrated in FIG. 9 and SEQ ID NO: 3 is intracellular domain of FcεRIγ. Finally, the 3′-UTR and Poly-A regions are present to provide stability to the RNA as well as initiation of translation. The longer poly-A region in this construct provides for an RNA construct with more stability and longer half-life time.

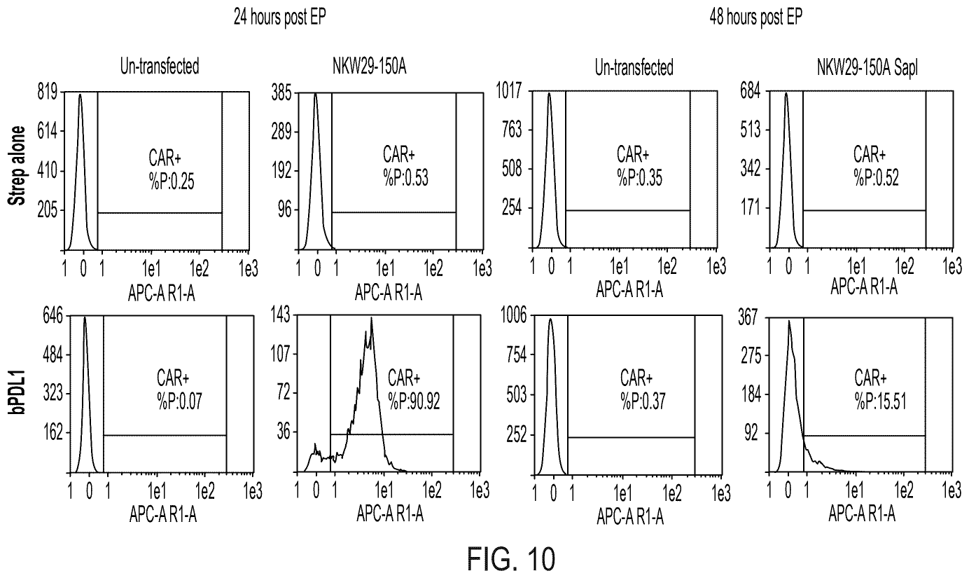

FIG. 10 illustrates time course of NKW29-150A PDL1 CAR expression in NK cells, 24 and 48 hours post electroporation. In vitro transcription was done using Sapl digested NKW29-150A DNA. NK cells were transfected with the in-vitro transcribed RNA (2 ug/1e6 cells). PDL1 expression was determined using flow cytometry and biotinylated PDL1/streptavidin APC.

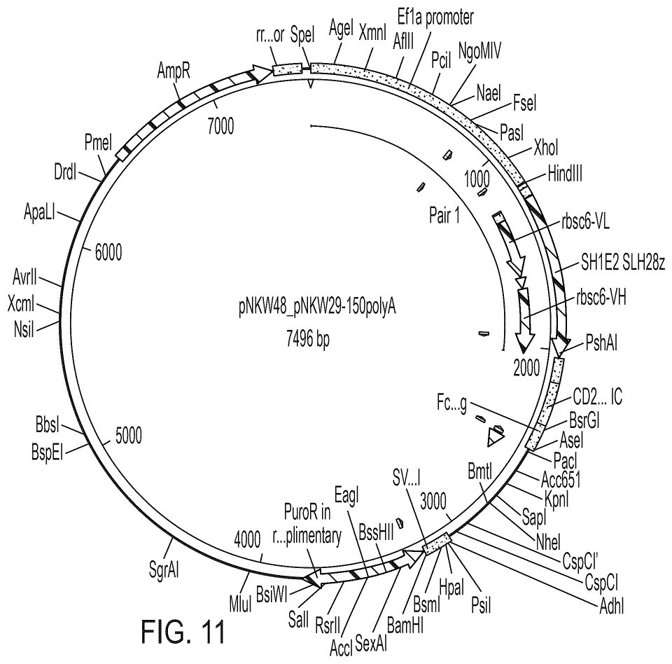

The vector map of the NKW29-150A construct is shown in FIG. 11. Finally, Table 3 below shows the different sequence portions of the NKW29-150A construct.

TABLE 3

| Construct name | NKW29-150A |
|---|---|
| Size | 7493 bp |
| SV40 polyA signal | 271-405 |
| Puromycin resistance gene | 1009-410 |
| Ampicillin resistance gene | 58-918 |
| rrnG terminator | 916-1051 |
| EF1α promoter | 2-1183 |
| T7 promoter | 1191-1209 |
| 5′UTR | 1210-1252 |
| Kozak sequence | 1246-1253 |
| CD64 signal peptide | 1254-1299 |
| Rbsc6-VL (variable light | 1300-1623 |
| chain) | |
| Linker | 1624-1683 |
| Rbsc6-VH (variable heavy | 1684-2037 |
| chain) | |
| CD28 hinge region | 2038-2154 |
| CD28 transmembrane domain | 2155-2235 |
| CD28 cytoplasmic domain | 2236-2358 |
| FceεRIγ cytoplasmic domain | 2359-2484 |
| 3′UTR Mus Musculus | 2492-2585 |
| hemoglobin alpha | |
| polyA | 2592-2741 |

In another embodiment, the inventors have disclosed the XL53-Tri-cistronic molecule, which comprises the recombinant nucleic acid construct of SEQ ID NO: 4. This molecule is similar to XL53 except for it co-expresses 3 genes: PDL1 CAR, CD16a and ER-retained I12. A P2A sequence and an EMCV IRES precede the CD16a and ER-IL2 genes respectively, allow independent translation of these genes. The main features of this construct are: (a) It expresses CD16a that engages in ADCC (antibody dependent cellular toxicity) and further triggers NK cell lysis of the target cells; and (b) It expresses IL-2, a cytokine that is a crucial growth factor for growth and cytotoxic activity of NK cells.

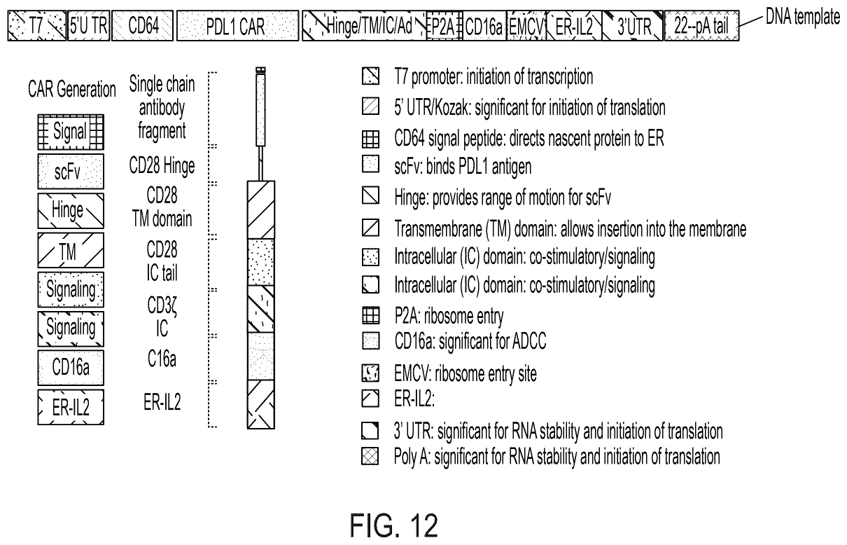

FIG. 12 illustrates generation of a chimeric antigen targeted at PDL1 protein, tricistronic XL53. Similar to the discussion in FIG. 1, the transcription of RNA is initiated by the T7 promoter. The 5′-UTR/Kozak region of nucleotides provides significant for initiation of translation. The nucleic acid sequence encoding the CD64 signal peptide is present in the 3′ end of the UTR/Kozak region, and it directs nascent protein to ER. This is followed by scFv region, which binds PDL1 antigen. A hinge region is present next to the scFv region, and that provides a range of motion to the scFv. The hinge region is followed by a transmembrane domain that allows insertion of the nucleotide construct into the membrane. This is followed by one or more intracellular domains comprising co-stimulatory and/or signaling elements. The co-stimulatory and/or signaling elements are followed by P2A for ribosome entry, CD16a which is significant for ADCC, EMCV which is the ribosome entry site, and ER-IL2. Finally, the 3′-UTR and Poly-A regions are present to provide stability to the RNA as well as initiation of translation. The longer poly-A region in this construct provides for an RNA construct with more stability and longer half-life time.

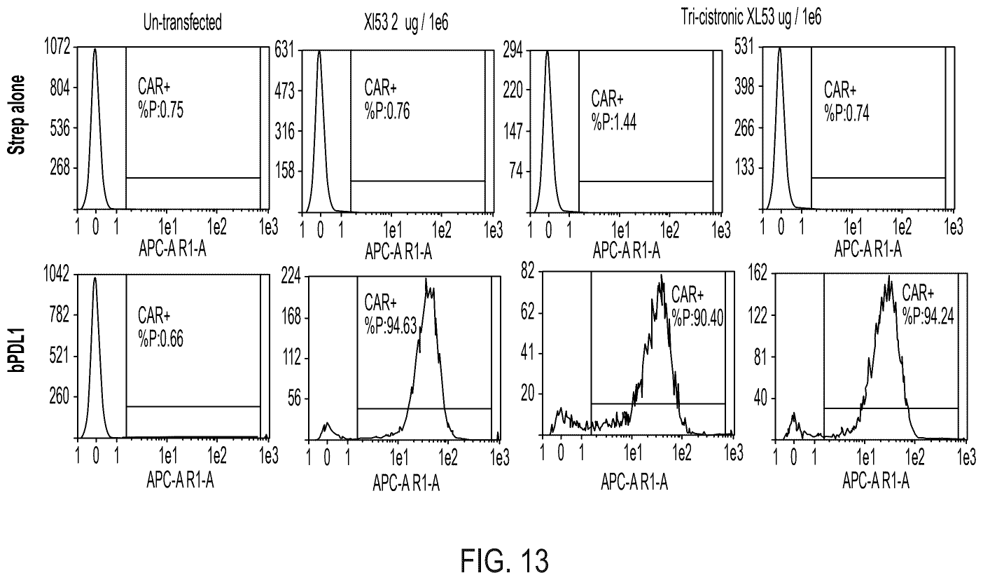

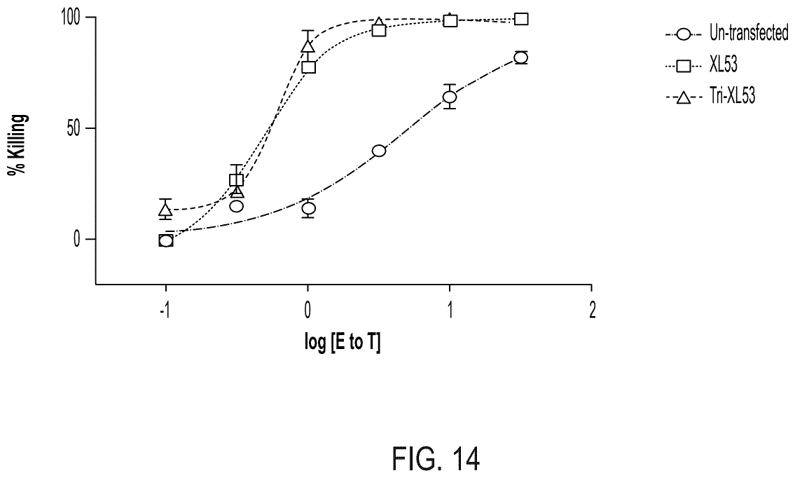

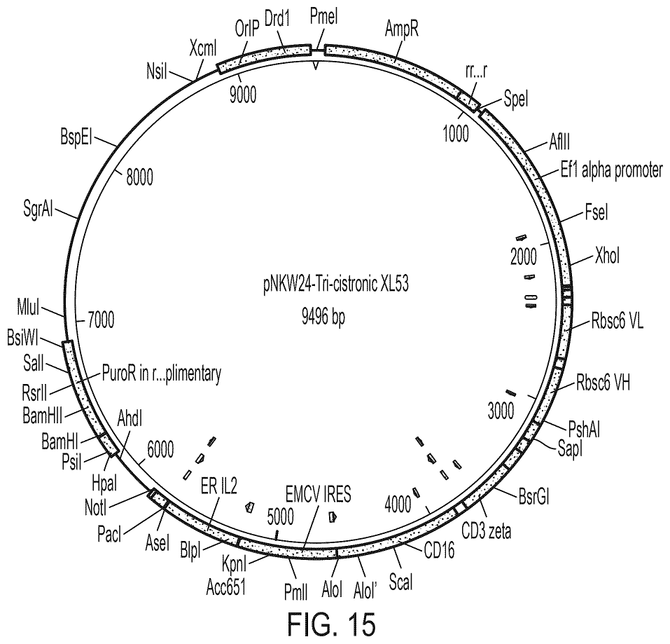

FIG. 13 illustrates XL53-tricistronic PDL1 CAR expression 24 hours post electroporation. The cytotoxic activity of CAR-infected cells on MS1 fLuc target cells are shown in FIG. 14. In this case, the transfected cells were mixed with target cells 2 hours post electroporation for overnight incubation. The vector map of the XL53-tricistronic construct is shown in FIG. 15. Finally, Table 4 below shows the different sequence portions of the XL53-tricistronic construct.

TABLE 4

| I. Construct name | XL-53 Tricistronic |
|---|---|
| Size | 9496 bp |
| Ampicillin resistance gene | 58-918 |
| rrnG terminator | 916-1051 |
| EF1a promoter | 1095-2276 |
| T7 promoter | 2284-2302 |
| 5′UTR | 2303-2345 |
| Kozak sequence | 2339-2346 |
| CD64 signal peptide | 2348-2392 |
| Rbsc6-VL (variable light | 2393-2716 |
| chain) | |
| Linker/spacer | 2717-2776 |
| Rbsc6-VH (variable heavy | 2777-3130 |
| chain) | |
| CD28 hinge region | 3131-3247 |
| CD28 transmembrane domain | 3248-3328 |
| CD28 cytoplasmic domain | 3329-3451 |
| CD3zeta cytoplasmic tail | 3452-3790 |
| P2A | 3791-3856 |
| FcγRIIIa (CD 16a) | 3857-4621 |
| EMCV IRES | 4622-5218 |
| ER IL2 | 5223-5705 |
| 3′UTR Mus Musculus | 5713-5806 |
| hemoglobin alpha | |
| polyA | 5807-5828 |
| SV40 polyA signal | 6145-6279 |
| Puromycin resistant gene | 6284-6883 |
| OriP | 8899-9465 |

Most currently available CAR technology uses viral vectors as a way of delivery of a DNA molecule to the cells. The viral DNA enters the nucleus and can integrate into the host genome. The inventors have developed a new approach that uses an RNA molecule because RNA only enters the cytoplasm and is ready to be translated. The inventors have overcome the degradation of RNA molecule problem by introducing several elements such as 5′ and 3′ UTR as well as a long poly-A to improve stability of the molecules disclosed herein.

Some variations to the inventive concept as contemplated by the inventors would be introduction of different 5′ or 3′ UTR elements that can improve stability of the RNA molecule. The construct can also be altered by addition (or swapping) of more co-stimulatory domains. Addition of other cytokine genes to the same construct (as a bi- or tri-cistronic) can also improve activity of the molecule.

In one embodiment, disclosed herein is a recombinant nucleic acid, comprising: a T7 promoter sequence portion, a 5′ untranslated (5′-UTR) sequence portion, a signal peptide sequence portion, a single chain antibody fragment sequence portion, a hinge region sequence portion, a transmembrane domain sequence portion, and one or more intracellular domain sequence portions. The signal peptide sequence portion further comprises a sequence encoding CD64. The RNA formed from the recombinant DNA nucleic acid is stabilized by a 5′-UTR sequence portion and/or a Kozak sequence. The Kozak sequence (or Kozak consensus sequence) is a nucleic acid motif that functions as the translation initiation site in most mRNA transcripts. It is regarded as the optimum sequence for initiating translation in eukaryotes, the sequence is an integral aspect of protein regulation. The sequence is generally defined as 5′-(gcc)gccRccAUGG-3′ where R indicates a purine (adenine or guanine). Of course, variations of the Kozak sequences are known to skilled artisans and contemplated herein by the inventors.

The single chain antibody fragment sequence portion of the recombinant nucleic acid comprises a sequence encoding for a single chain variable fragment that is adapted to bind PDL1 antigen. The hinge portion plays the role of providing range of motion for the single chain antibody fragment sequence portion. The transmembrane domain sequence portion enables insertion of the recombinant nucleic acid to a membrane. The intracellular domain sequence portion comprises co-stimulatory or signaling sequence portions such as CD28, CD32, and/or FcεRIγ. The intracellular domain sequence portions are selected to provide enhanced cytotoxic activity against tumor cells. The 3′-UTR region towards the 3′ end of the recombinant nucleic acid provides stability to the RNA and initiation of translation. Furthermore, a poly-A sequence portion may be present for additional stability reason. In some embodiments, the poly-A sequence portion comprises at least 150 adenine nucleotides. In some embodiments, the recombinant nucleic acid may be tri-cistronic—in other words, the nucleic acid may have sequence encoding for PDL1-CAR, CD16a, and ER-IL2.

In another aspect of the instant disclosure, provided herein are modified NK cells comprising one or more nucleic acids encoding: a T7 promoter sequence portion, a 5′ untranslated (5′-UTR) sequence portion, a signal peptide sequence portion, a single chain antibody fragment sequence portion, a hinge region sequence portion, a transmembrane domain sequence portion, and one or more intracellular domain sequence portions; wherein the nucleic acid sequences are operably linked to each other as a single polynucleotide.

Natural killer (NK) cells are cells of the immune system that kill target cells in the absence of a specific antigenic stimulus, and without restriction according to major histocompatibility complex (MHC) class. NK cells are characterized by the presence of CD56 and the absence of CD3 surface markers. Endogenous NK cells are generally heterogeneous populations of cells within which NK cells have been enriched. Endogenous NK cells may be intended for autologous or allogeneic treatment of a patient.

As used herein, “immunotherapy” refers to the use of NK cells modified or unmodified, naturally occurring or modified NK cell or T-cell, whether alone or in combination, and which are capable of inducing cytotoxicity when contacting a target cell.

### Treatment of Cancer

Provided herein are methods of treating a cancer or a tumor in a subject, the method comprising administering to the subject a therapeutically effective amount of the modified NK cells as disclosed above or a composition comprising modified NK cells as disclosed above to a patient in need thereof. The administration is contemplated to treat the cancer, reduces the size of the tumor in the subject, or reduce cancer metastasis in the subject.

The term “cancer” refers to all types of cancer, neoplasm, or malignant tumors found in mammals, including leukemia, carcinomas and sarcomas. Exemplary cancers include cancer of the brain, breast, cervix, colon, head & neck, liver, kidney, lung, non-small cell lung, melanoma, mesothelioma, ovary, sarcoma, stomach, uterus and Medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

The terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms subject, patient, individual, etc. are not intended to be limiting and can be generally interchanged. That is, an individual described as a patient does not necessarily have a given disease, but may be merely seeking medical advice. As used throughout, a subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient, individual and subject may be used interchangeably and these terms are not intended to be limiting. That is, an individual described as a patient does not necessarily have a given disease, but may be merely seeking medical advice. The terms patient or subject include human and veterinary subjects.

“Administration” or “administering,” as used herein, refers to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional or intracranial injection), transdermal, topical, buccal, rectal, vaginal, nasal, ophthalmic, via inhalation, and implants. Optionally, the NK cells are administered parenterally. Optionally, the NK cells are administered intravenously. Optionally, the NK cells are administered peritumorally.

The modified NK cells as disclosed herein can be administered to a subject by absolute numbers of cells, e.g., said subject can be administered from about 1000 cells/injection to up to about 10 billion cells/injection, such as at about, at least about, or at most about, 1×10¹⁰, 1×10⁹, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK cells per injection, or any ranges between any two of the numbers, end points inclusive. Optionally, from 1×10⁸ to 1×10¹⁰ cells are administered to the subject. Optionally, the cells are administered one or more times weekly for one or more weeks. Optionally, the cells are administered once or twice weekly for 1, 2, 3, 4, 5, 6, 7, 8, 9, 10 or more weeks.

In another embodiment, the total dose may also calculated by m² of body surface area. The subject may be administered from about 1000 cells/injection/m² to up to about 10 billion cells/injection/m², such as at about, at least about, or at most about, 1×10¹⁰/m², 1×10⁹/m², 1×10⁸/m², 1×10⁷/m², 5×10⁷/m², 1×10⁶/m², 5×10⁶/m², 1×10⁵/m², 5×10⁵/m², 1×10⁴/m², 5×10⁴/m², 1×10³/m², 5×10³/m² (and so forth) NK cells per injection, or any ranges between any two of the numbers, end points inclusive. Optionally, from 1×10³ to 1×10¹⁰, per m² of the NK cells are administered to the subject. Optionally, 2×10⁹ per m², of the NK cells are administered to the subject.

Optionally, NK cells can be administered to such individual by relative numbers of cells, e.g., said individual can be administered about 1000 cells to up to about 10 billion cells per kilogram of the individual, such as at about, at least about, or at most about, 1×10¹⁰, 1×10⁹, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK® cells per kilogram of the individual, or any ranges between any two of the numbers, end points inclusive.

In some embodiments, NK cells are administered in a composition comprising NK cells and a medium, such as human serum or an equivalent thereof. The medium may comprise human serum albumin and/or human plasma. Optionally, the medium comprises about 1% to about 15% human serum or human serum equivalent. Optionally, the medium comprises about 1% to about 10% human serum or human serum equivalent. Optionally, the medium comprises about 1% to about 5% human serum or human serum equivalent. Optionally, the medium comprises about 2.5% human serum or human serum equivalent. Optionally, the serum is human AB serum. Optionally, a serum substitute that is acceptable for use in human therapeutics is used instead of human serum. Such serum substitutes may be known in the art. Optionally, NK cells are administered in a composition comprising NK cells and an isotonic liquid solution that supports cell viability. Optionally, NK cells are administered in a composition that has been reconstituted from a cryopreserved sample.

According to the methods provided herein, the subject is administered an effective amount of one or more of the agents provided herein. The terms effective amount and effective dosage are used interchangeably. The term effective amount is defined as any amount necessary to produce a desired physiologic response (e.g., reduction of inflammation). Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 22nd Edition, Gennaro, Editor (2012), and Pickar, Dosage Calculations (1999)).

The provided methods may be further combined with other tumor therapies such as radiotherapy, surgery, hormone therapy and/or immunotherapy. Thus, the provided methods can further include administering one or more additional therapeutic agents to the subject. Suitable additional therapeutic agents include, but are not limited to, analgesics, anesthetics, analeptics, corticosteroids, anticholinergic agents, anticholinesterases, anticonvulsants, antineoplastic agents, allosteric inhibitors, anabolic steroids, antirheumatic agents, psychotherapeutic agents, neural blocking agents, anti-inflammatory agents, antihelmintics, antibiotics, anticoagulants, antifungals, antihistamines, antimuscarinic agents, antimycobacterial agents, antiprotozoal agents, antiviral agents, dopaminergics, hematological agents, immunological agents, muscarinics, protease inhibitors, vitamins, growth factors, and hormones. The choice of agent and dosage can be determined readily by one of skill in the art based on the given disease being treated. Optionally, the additional therapeutic agent is octreotide acetate, interferon, pembrolizumab, glucopyranosyl lipid A, carboplatin, etoposide, or any combination thereof.

In some embodiments, the additional therapeutic entity may be selected from the group consisting of a viral cancer vaccine, a bacterial cancer vaccine, a yeast cancer vaccine, N-803, an antibody, a stem cell transplant, and a tumor targeted cytokine.

Optionally, the additional therapeutic agent is a chemotherapeutic agent. A chemotherapeutic treatment regimen can include administration to a subject of one chemotherapeutic agent or a combination of chemotherapeutic agents. Chemotherapeutic agents include, but are not limited to, alkylating agents, anthracyclines, taxanes, epothilones, histone deacetylase inhibitors, inhibitors of Topoisomerase I, inhibitors of Topoisomerase II, kinase inhibitors, monoclonal antibodies, nucleotide analogs and precursor analogs, peptide antibiotics, platinum-based compounds, retinoids, and vinca alkaloids and derivatives. Optionally, the chemotherapeutic agent is carboplatin.

Combinations of agents or compositions can be administered either concomitantly (e.g., as a mixture), separately but simultaneously (e.g., via separate intravenous lines) or sequentially (e.g., one agent is administered first followed by administration of the second agent). Thus, the term combination is used to refer to concomitant, simultaneous, or sequential administration of two or more agents or compositions. The course of treatment is best determined on an individual basis depending on the particular characteristics of the subject and the type of treatment selected. The treatment, such as those disclosed herein, can be administered to the subject on a daily, twice daily, bi-weekly, monthly, or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A modified NK cell comprising one or more nucleic acids encoding:
a 5′ untranslated (5′-UTR) sequence portion, a signal peptide sequence portion, a single chain antibody fragment sequence portion, a hinge region sequence portion, a transmembrane domain sequence portion, and one or more intracellular domain sequence portions;
wherein the nucleic acid sequences are operably linked to each other as a single polynucleotide.

2. The modified NK cell of claim 1, wherein the cell specifically targets a tumor cells.

3. The modified NK cell of claim 1, wherein the modified NK cell specifically targets PDL1 or other tumor antigens.

4. The modified NK cell of claim 1, wherein the signal peptide sequence portion further comprises a sequence encoding CD64.

5. The modified NK cell of claim 1, wherein the single chain antibody fragment sequence portion comprises a sequence encoding for a single chain variable fragment that is adapted to bind PDL1 antigen or other tumor antigens.

6. The modified NK cell of claim 1, wherein the hinge sequence portion provides range of motion for the single chain antibody fragment sequence portion.

7. The modified NK cell of claim 1, wherein the transmembrane domain sequence portion enables insertion of the recombinant nucleic acid to a membrane.

8. The modified NK cell of claim 1, wherein the intracellular domain sequence portion comprises co-stimulatory or signaling sequence portions.

9. The modified NK cell of claim 1, wherein the intracellular domain sequence portion comprises CD28 and/or CD3ζ and/or FcεRIγ.

10. The modified NK cell of claim 1, wherein the intracellular domain sequence portion provides enhanced cytotoxic activity against tumor cells.

11. The modified NK cell of claim 1, further comprising a 3′-untranslated region (3′-UTR)

12. The modified NK cell of claim 1, wherein the 3′-UTR sequence portion provides RNA stability and initiation of translation.

13. The modified NK cell of claim 1, further comprising a poly-A sequence portion

14. The modified NK cell of claim 1, wherein the poly-A sequence portion comprises at least 150 adenine nucleotides.

15. The modified NK cell of claim 1, wherein the poly-A sequence portion provides RNA stability and initiation of translation.

16. The modified NK cell of claim 1, wherein the vector targets a tumor antigen.

17. The modified NK cell of claim 1, further comprising a sequence portion encoding CD16a.

18. The modified NK cell of claim 1, further comprising a sequence portion encoding ER-IL2.

19. A composition comprising the modified NK cell of claim 1 and a pharmaceutically acceptable excipient.

20. A method of treating a cancer or a tumor in a subject, the method comprising administering to the subject a therapeutically effective amount of the modified NK cells of claim 1, wherein administration treats the cancer or reduces the size of the tumor in the subject.


SEQUENCE LISTING (4 sequences)

SEQ ID NO: 1 - DNA, 7584 bp, synthetic construct
  misc_feature 1..7584: note = XL53
  source 1..7584: mol_type = other DNA
agctgccttctgcggggcttgccttctggccatgcccttcttctctcccttgcacctgtacctcttggtctttgaataaagcctgagtaggaagaaaaaaaaaaaaaaaaaaaaaagcaggtggcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctatcctcatgcatatacagtcagcatatgatacccagtagtagagtgggagtgctatcctttgcatagcccatcccgcccctaactccgcccagttccgcccattctccgcctcatggctgactaattttttaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctttaatacgactcactatagggaaataagagagaaaagaagagtaagaagaaatataagagccaccatgtggtttcttactacattgctgttgtgggtcccggtggacggtaacatccagatgacccagtctccatcttctgtgtctgcatctgtaggagacagagtcaccatcacttgtcgggcgagtcaggatattagccgctggttagcctggtatcagcagaaaccagggaaagcccctaaactcctgatctatgctgcatccagtttgcaaagtggggtcccatcgaggttcagcggcagtggatctgggacagatttcgctctcactatcagcagcctgcagcctgaagattttgcaacttactattgtcaacaggctgacagtcgtttctcgatcaccttcggccaagggacacgactggagattaaaggcggcggaggaagcggaggcggaggatctgggggcggaggctctggcggagggggatctgaggtgcagctggtgcagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatacagtacgcagactctgtgaagggccgattcaccatctccagagacaatgccaagaactcactgtatctgcaaatgaacagcctgagagacgaggacacggctgtgtattactgtgcgagaggggactactactacggtatggacgtctggggccaagggaccacggtcaccgtgagctcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaatta

SEQ ID NO: 2 - DNA, 7709 bp, synthetic construct
  misc_feature 1..7709: note = XL53-150A
  source 1..7709: mol_type = other DNA
tcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctatcctcatgcatatacagtcagcatatgatacccagtagtagagtgggagtgctatcctttgcatagcccatcccgcccctaactccgcccagttccgcccattctccgcctcatggctgactaattttttaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctttaatacgactcactatagggaaataagagagaaaagaagagtaagaagaaatataagagccaccatgtggtttcttactacattgctgttgtgggtcccggtggacggtaacatccagatgacccagtctccatcttctgtgtctgcatctgtaggagacagagtcaccatcacttgtcgggcgagtcaggatattagccgctggttagcctggtatcagcagaaaccagggaaagcccctaaactcctgatctatgctgcatccagtttgcaaagtggggtcccatcgaggttcagcggcagtggatctgggacagatttcgctctcactatcagcagcctgcagcctgaagattttgcaacttactattgtcaacaggctgacagtcgtttctcgatcaccttcggccaagggacacgactggagattaaaggcggcggaggaagcggaggcggaggatctgggggcggaggctctggcggagggggatctgaggtgcagctggtgcagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatacagtacgcagactctgtgaagggccgattcaccatctccagagacaatgccaagaactcactgtatctgcaaatgaacagcctgagagacgaggacacggctgtgtattactgtgcgagaggggactactactacggtatggacgtctggggccaagggaccacggtcaccgtgagctcaattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagggtaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagctctaggctagcccaggcctcgccctccagc

SEQ ID NO: 3 - DNA, 7493 bp, synthetic construct
  misc_feature 1..7493: note = NKW29
  source 1..7493: mol_type = other DNA
aggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctttaatacgactcactatagggaaataagagagaaaagaagagtaagaagaaatataagagccaccatgtggtttcttactacattgctgttgtgggtcccggtggacggtaacatccagatgacccagtctccatcttctgtgtctgcatctgtaggagacagagtcaccatcacttgtcgggcgagtcaggatattagccgctggttagcctggtatcagcagaaaccagggaaagcccctaaactcctgatctatgctgcatccagtttgcaaagtggggtcccatcgaggttcagcggcagtggatctgggacagatttcgctctcactatcagcagcctgcagcctgaagattttgcaacttactattgtcaacaggctgacagtcgtttctcgatcaccttcggccaagggacacgactggagattaaaggcggcggaggaagcggaggcggaggatctgggggcggaggctctggcggagggggatctgaggtgcagctggtgcagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatacagtacgcagactctgtgaagggccgattcaccatctccagagacaatgccaagaactcactgtatctgcaaatgaacagcctgagagacgaggacacggctgtgtattactgtgcgagaggggactactactacggtatggacgtctggggccaagggaccacggtcaccgtgagctcaattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgaagatccaggtccgaaaggccgccatcaccagctacgagaagtctgatggcgtgtacaccggcctgagcaccagaaaccaggaaacctacgagacactgaagcacgagaagcccccccagtgattaattaagctgccttctgcggggcttgccttctggccatgcccttcttctctcccttgcacctgtacctcttggtctttgaataaagcctgagtaggaagggtaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagctctaggctagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctatcctcatgcatatacagtcagcatatgatacccagtagtagagtgggagtgctatcctttgcatagcccatcccgcccctaactccgcccagttccgcccattctccgcctcatggctgactaattttttaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtg

SEQ ID NO: 4 - DNA, 9496 bp, synthetic construct
  misc_feature 1..9496: note = XL53-Tri-cistronic
  source 1..9496: mol_type = other DNA
tgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctttaatacgactcactatagggaaataagagagaaaagaagagtaagaagaaatataagagccaccatgtggtttcttactacattgctgttgtgggtcccggtggacggtaacatccagatgacccagtctccatcttctgtgtctgcatctgtaggagacagagtcaccatcacttgtcgggcgagtcaggatattagccgctggttagcctggtatcagcagaaaccagggaaagcccctaaactcctgatctatgctgcatccagtttgcaaagtggggtcccatcgaggttcagcggcagtggatctgggacagatttcgctctcactatcagcagcctgcagcctgaagattttgcaacttactattgtcaacaggctgacagtcgtttctcgatcaccttcggccaagggacacgactggagattaaaggcggcggaggaagcggaggcggaggatctgggggcggaggctctggcggagggggatctgaggtgcagctggtgcagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatacagtacgcagactctgtgaagggccgattcaccatctccagagacaatgccaagaactcactgtatctgcaaatgaacagcctgagagacgaggacacggctgtgtattactgtgcgagaggggactactactacggtatggacgtctggggccaagggaccacggtcaccgtgagctcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggatctggagctactaacttcagcctgctgaagcaggctggagacgtggaggagaaccctggacctatgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtgaaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataaccgccaccatgtaccggatgcagctgctgagctgtatcgccctgtctctggccctcgtgaccaacagcgcccctaccagcagcagcaccaagaaaacccagctgcagctggaacatctgctgctggacctgcagatgatcctgaacggcatcaacaactacaagaaccccaagctgacccggatgctgaccttcaagttctacatgcccaagaaggccaccgaactgaaacatctgcagtgcctggaagaggaactgaagcccctggaagaagtgctgaacctggcccagagcaagaacttccacctgaggcccagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgaccggctccgagaaggacgagctgtgattaattaagctgccttctgcggggcttgccttctggccatgcccttcttctctcccttgcacctgtacctcttggtctttgaataaagcctgagtaggaagaaaaaaaaaaaaaaaaaaaaaagcaggtggcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctatcctcatgcatatacagtcagcatatgatacccagtagtagagtgggagtgctatcctttgcatagcccatcccgcccctaactccgcccagttccgcccattctccgcctcatggctgactaattttttaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaact